(19) United States
(12) Patent Application Publication
(10) Pub. No.: US 2023/0069525 A1
(43) Pub. Date: Mar. 2, 2023

(54) ELECTROSURGICAL GENERATOR HAVING AN HF HIGH-VOLTAGE MULTILEVEL INVERTER

(71) Applicant: OLYMPUS WINTER & IBE GMBH, Hamburg (DE)

(72) Inventors: Jelle DIJKSTRA, Berlin (DE); Thomas FÄHSING, Berlin (DE); Daniel RAMIN, Nuthetal (DE); Stefan DIETRICH, Potsdam (DE); Thomas PREZEWOWSKY, Teltow (DE); Stefan SCHIDDEL, Stahnsdorf (DE); Dimitri BECKER, Berlin (DE)

(73) Assignee: OLYMPUS WINTER & IBE GMBH, Hamburg (DE)

(21) Appl. No.: 17/890,901

(22) Filed: Aug. 18, 2022

Related U.S. Application Data
(60) Provisional application No. 63/237,397, filed on Aug. 26, 2021.

(51) Int. Cl. H02M 7/483; H02M 7/49

(52) U.S. Cl. CPC H02M 7/483; H02M 7/49; H02M 7/487

(57) ABSTRACT

An electrosurgical generator for an electrosurgical instrument includes a DC voltage supply and a high-voltage inverter that generates a high-frequency AC voltage having a variable voltage and frequency that is output at an output for the connection of the electrosurgical instrument. The inverter is configured as a multilevel inverter and includes a plurality of inverter cells connected in a cascaded manner that are driven by a control device. Thanks to the cascading, switching losses incurred in the power semiconductors are reduced, both in terms of value (through the divided and thus lower voltage) and in terms of frequency (through the reduced switching frequency).

The invention relates to an electrosurgical generator that is designed to output a high-frequency AC voltage to an electrosurgical instrument. It comprises a DC voltage supply and a high-voltage inverter that is fed from the DC voltage supply and generates a high-frequency AC voltage that is applied at an output for the connection of the electrosurgical instrument.

In electrosurgery, high-frequency AC current is used in particular to cut or slice through tissue and to excise body tissue within the meaning of a thermal resection (what is known as an electrical scalpel). The operating principle is based on heating the tissue to be cut. One advantage of this is that, at the same time as the cutting, it is also possible to stem bleeding by closing the affected vessels (coagulation). Considerable powers are required for this purpose, specifically at frequencies of 200 kHz or higher up to 4000 kHz, typically around 400 kHz. Body tissue behaves like an ohmic resistance at these frequencies. However, the specific resistance is strongly dependent on the type of tissue, meaning that the specific resistances of muscles, fat or bones differ greatly from one another, specifically by up to a factor of 1000. This means that, during operation, the load impedance of the electrical scalpel may change quickly and greatly depending on the tissue to be cut, as far as a virtual short circuit. This places special and unique requirements on the electrosurgical generator and its high-voltage supply. There is in particular a need for fast voltage control, suitable for high voltages in the range of a few kilovolts, and high frequencies in a wide range from typically between 200 kHz and up to 4 MHz.

Depending on the tissue and the resulting impedance, the currents vary between a few milliamperes and several amperes, specifically in a highly dynamic manner within a very short time. The waveform of the AC voltage that is output may be continuously sinusoidal or may be modulated with a crest factor of up to 10 at modulation frequencies of up to around 20 kHz.

In order to meet these unique requirements, electrosurgical generators are typically structured such that they have an inverter for supplying power to the electrosurgical instrument, to which rectified current from the grid is supplied with a different voltage. The inverter is in turn typically configured as a freely oscillating single-ended generator having an LC resonant circuit (see, in FIG. 13 regarding the prior art, the block 114 highlighted by dashed lines, which is fed by a power supply unit 112 in order to supply power to an instrument 116). This structure is proven (for example: EP 2 514 380 B 1). However, more recently, an increasing number of modulated modes in which electrosurgical instruments are driven in a clocked manner have become significant. Examples of such modes are contained in FIGS. 9a to 9e. The electrosurgical generators may thus, for example in a cutting mode for cutting tissue, continuously output a voltage of for example 600 volts (RMS value) (FIG. 9a) and, in a coagulation mode, output modulated high voltage with a peak voltage of up to 4500 V, but in the manner of intervals with a small duty cycle (FIG. 9e). Various further modes with other types of voltage/time profiles may be set here (see FIG. 9b-d). Modes with a small duty cycle and large, fast voltage jumps in particular place high demands on electrosurgical generators.

Electrosurgical generators having a parallel resonant circuit do make it possible to generate the required high frequencies, but have a number of disadvantages. First of all, efficiency is low due to high losses. In addition, large reactive currents occur in the parallel resonant circuit, thereby necessitating larger components and additionally worsening efficiency at low power. Furthermore, the output frequency is load-dependent, as is the crest factor, this being inappropriate for highly modulated modes. The regulation of the output voltage is comparatively slow, meaning that matching to changed load impedances is only poor.

In other fields, such as in the case of audio amplifiers, it is known, as power stage, to provide inverters that are structured in accordance with digital amplifier technology, what are known as class D amplifiers. However, this type of structure, with its output frequency in the low-frequency range, is not used for high-frequency applications such as for electrosurgical generators. This is because power losses that arise during changeover of the power semiconductors increase linearly with frequency and even quadratically with voltage, which would lead for example, at for instance six times the frequency and also six times the voltage, to an unacceptable increase in the power loss factor to (6³=) 216. This cannot be justified either with regard to the losses in the power semiconductors or for efficiency aspects.

The invention is based on the object of improving an electrosurgical generator of the type mentioned at the outset with regard to its operating behavior, specifically in particular in the case of modulated modes.

The solution according to the invention lies in the features of the independent claim. Advantageous developments are the subject of the dependent claims.

In the case of an electrosurgical generator that is designed to output a high-frequency AC voltage to an electrosurgical instrument, comprising a DC voltage supply and a high-voltage inverter that is fed from the DC voltage supply and generates a high-frequency AC voltage having a variable voltage and frequency that is applied to an output for the connection of the electrosurgical instrument, provision is made, according to the invention, that the inverter is configured as a multilevel inverter and comprises a plurality of inverter cells connected in a cascaded manner that are driven by a control device.

The core concept of the invention is the idea of dividing the high voltage and high frequency typically to be output by the electrosurgical generator over multiple inverter cells. The switching losses incurred in the power semiconductors of the individual inverter cell are thereby reduced. This applies both in terms of value (through the divided and thus lower voltage) and in terms of frequency (through the reduced switching frequency). Since in particular power loss increases quadratically with voltage, thanks to the cascading, it is thereby possible to achieve disproportionate alleviation of the inverter cells with the multilevel inverter according to the invention. In the case of an arrangement of ten inverter cells, this thus results for example, for each of the inverter cells, in only one tenth of the switching procedures for ( 1/10)² corresponding to one hundredth of the power loss. However, advantages result not only in relation to voltage and frequency strength, but also in relation to dynamic range. This is because, with the inverter cells, the output AC voltage is able to be adapted quickly to changes, in particular jumps in the load impedance, wherein the curve form is able to be chosen practically freely. The output AC voltage is thus also able to be modulated to a great extent, including sudden and high voltage peaks, without overloading the inverter cells with their power semiconductors. It is thus possible in particular also to achieve stable output voltages in highly pulsed modes, with a negligible predefinable crest factor, even with small duty cycles.

A few terms that are used will first of all be explained below:

In the field of electrosurgical generators, “high-frequency” frequencies are typically understood to be in the range from 200 kHz to 4000 kHz. Optionally, in advantageous embodiments, the ultrasonic range may also be covered. The ultrasonic range is understood to mean a frequency range between 20 kHz and 200 kHz.

“High-voltage” is typically understood to mean voltages up to 10 kV, preferably up to 5000 V.

The power provided by the electrosurgical generator is typically in the range between 1 and 500 watts, wherein the load impedance may vary greatly, and output voltage and power output may accordingly likewise change greatly and quickly.

Multilevel inverters are inverters generating an AC voltage from DC voltage that are able to generate more than two voltage levels other than zero at their output.

The inverter cells expediently each have potential decoupling at output. The invention makes use of the fact here that AC voltage is by definition present at the output of the inverter cells, meaning that it is possible to achieve reliable potential isolation of the voltages ultimately output by the inverter cells using simple and inexpensive transformers in a manner requiring little outlay (in comparison with isolated individual DC voltage sources as would be required at input).

It is preferable, for potential decoupling purposes, for provision to be made for transformers at the output of the inverter cells. The respective transformer on each of the inverter cells thus ensures that the output voltage ultimately output by the respective inverter cell is potential-free. Advantageously, the transformers are connected at the output of the respective inverter cell with their respective primary side, wherein secondary sides of the transformers are chained in order to sum secondary voltages of the respective transformers, wherein the summed voltage is channeled via an output line to the output for the connection of the electrosurgical instrument.

This thus results overall in an improvement of the switching behavior in combination with a considerable reduction in expenditure. Although the arrangement of the transformers at the output of the inverter cells means that it is no longer possible to output DC voltage, this is not a disadvantage—as the invention has also identified—in the field of electrosurgical generators, but rather an advantage.

This is because patient safety is thus additionally increased since the entire inverter arrangement loses its intrinsic capability to output DC current (which is dangerous to patients). The potential decoupling, in particular transformers, at the output of the inverter cells in this respect act as a further protective shield for the patient.

The transformers are preferably each provided with a transformer unit as preamplifier for stepping up the voltage. The voltage output by the inverter cells is thus able to be amplified, with at the same time the currents flowing in the output line being reduced. It is particularly expedient for the transformer to be configured to be structurally integrated with the respective transformer unit. This allows a particularly inexpensive and space-saving combination of the two functions of galvanic isolation, on the one hand, and amplifying the voltage, on the other hand.

The inverter cells are preferably fed from a respective DC voltage source. The DC voltage sources may in this case be isolated from one another or separated from one another in terms of potential. However, this is not necessary, but rather provision may optionally be made for them to be linked via a reference potential. Complex potential isolation of the DC voltage sources at the input of the inverter cells is thus unnecessary.

However, provision may also be made that a plurality of, at least two, groups of inverter cells are provided, wherein the inverters of the respective group are supplied jointly by one DC voltage source. Combining into groups thus allows efficient utilization of the DC voltage sources, which reduces expenditure. Other advantages may however also be achieved with the common supply, as explained below. A “group” should be understood here to mean that it comprises at least one inverter cell.

The DC voltage sources are expediently fed jointly from the DC voltage supply. The DC voltage supply may in particular comprise a DC link circuit that is fed with DC for example by a power supply unit or directly by an external means. Complex provision of separate, even potentially isolated DC voltage sources for the inverter cells is thus no longer necessary. This not only simplifies the provision of the DC voltage required for the operation of the respective inverter cells. It also allows a considerable structural simplification. This is because, during operation of a multilevel inverter, there may be states in which the direction of the power flow reverses in at least one of the inverter cells, that is to say power is fed back into the DC voltage source. This requires what are known as bidirectional DC voltage sources, which are more complex than usual ones. If a large number of DC voltage sources are required, for example one for each inverter, then expenditure increases considerably. However, since the invention makes it possible no longer to have to isolate the DC voltage sources, but rather to be able to switch them together, any backflow of power through one of the inverter cells into the DC voltage source is compensated for by another one of the inverter cells with a regular power flow in the forward direction. This is all the more true in the case of a combination of the inverter cells to form groups of multiple inverter cells. This ultimately results in barely any or even no interfering backflow of power. However, even when a backflow of power occurs, it is then enough to configure only one DC voltage source to be bidirectional, rather than a large number of them as before.

Advantageously, provision is made for a plurality of, at least two groups of inverter cells, wherein the groups are supplied with DC voltage of different values, wherein preferably one group of the plurality of groups is supplied with a DC voltage that is at least twice as high as another group of the plurality of groups. A “group” should be understood here to mean that it comprises at least one inverter cell. By supplying a group of inverter cells with a different DC voltage, it is possible to increase the maximum number of voltage levels able to be output in comparison with an identical number of inverter cells that are all fed with the same voltage. This makes it possible to achieve finer gradation of the output AC voltage. Provision may furthermore be made for the driving of the inverter cells to be designed so as to further reduce the number of switching procedures, which in turn contributes to reducing power loss. Provision is expediently made for three or more groups of inverter cells, wherein the value of the DC voltage supplied to them is in each case different. Provision may advantageously be made that the different DC voltages that are supplied in particular follow a geometric sequence. A division of the DC voltage that is supplied preferably has the ratio 1:3:9, in order thus to be able to achieve the highest possible number of different levels with three groups.

In this case, provision is advantageously made for in each case at least one in particular ratiometric DC-to-DC converter for supplying at least one of the groups with a different voltage. The ratio of the DC voltage that is supplied thus remains constant even in the event of fluctuations in the absolute value of the DC voltage. It is particularly expedient for the DC-to-DC converter to be bidirectional, for example to be able to convert 12 volts DC to 48 volts DC or vice versa. This enables flexible use in particular in environments with a DC supply, for example in conventional vehicles with a 12 volt on-board power supply or in modern vehicles with a powerful 48 volt on-board power supply. Depending thereon, the bidirectional DC-to-DC converter then performs a step-up conversion from 12 volts to 48 volts or a step-down conversion from 48 volts to 12 volts.

The invention thus manages, by way of a measure that appears surprisingly simple at first glance, in a simple and expedient manner, to solve the difficulties that occur in multilevel inverters with regard to the DC voltage sources, specifically their potential isolation and their bidirectional capability (that is to say including power consumption capability), in one go.

The DC voltage sources of the inverter cells are advantageously galvanically coupled. The galvanic coupling enables simple and overall less complex connection of the DC voltage sources to the inverter cells. This in particular also makes it possible to achieve the concept whereby a single source is provided in order to supply the multiplicity of inverter cells. The DC link circuit of the electrosurgical generator expediently operates here as DC voltage source in each case. This enables a conceptually simple and robust structure.

It is particularly expedient for the DC voltage supply to be designed as a fixed voltage supply, in particular to comprise a DC link circuit having a fixed voltage level. The in particular ratiometric DC-to-DC converter may optionally be connected thereto. Such a fixed voltage supply enables a significant simplification in comparison with types of structure that require a complex DC voltage with a changeable voltage and accordingly require a DC link circuit with a changeable voltage. A supply with a fixed-value DC voltage is sufficient for the invention, and the multilevel inverter according to the invention takes on all of the remaining voltage adjustment for the output voltage range, which covers hundreds or thousands of volts.

The DC voltage supply may be arranged internally or externally. It may be configured as a power supply unit for connection to a supply grid, in particular a three-phase or AC grid, or else be designed to be fed directly with DC voltage. The last case is advantageous in particular for mobile applications in vehicles (24 volts DC feed, also 48 volts DC in modern vehicles) or in other environments that are supplied with DC (for example with 48 volts DC).

With regard to the design of the inverter cells as such, the invention is not restricted to one type of structure. Provision may thus be made that the inverter cells are also configured for example with a type of structure with neutral point clamping or with a type of structure with a floating capacitor. Cells with neutral point clamping are of comparatively simple design, in particular in terms of their expedient configuration with clamp diodes. A three-level inverter thus requires only two diodes. More levels are possible, as a result of which it is possible to achieve a finer gradation and the voltage loading for each diode is reduced. However, the number of diodes required increases quadratically with the number of levels, which limits the number of levels for practical reasons. In this regard, inverter cells of the type of structure with a floating capacitor are more appropriate. These have similar advantages to those with diodes, but the number of capacitors required increases to a lesser extent with additional levels. However, a configuration of the inverter cells in which they are connected in series is preferred. They are in particular each configured in an H-bridge configuration. It is thereby possible to make provision for any desired number per se of inverter cells, as a result of which the voltage loading incumbent on each of the inverter cells decreases in a manner inversely proportional to the number of inverter cells. This reduces not only voltage losses through the power semiconductors, but also their switching losses. In the case of the cascaded arrangement, the number of switching procedures per inverter cell is also reduced, this likewise contributing to reducing switching losses.

In order to drive the multilevel inverter with its inverter cells, provision is expediently made for a control signal generator that is designed to generate a reference signal for driving the multilevel inverter. This makes it possible to achieve precise control of the type of AC voltage that is output, which is a significant advantage in particular in relation to the ability to set different modes. The reference signal is expediently a pattern for the AC voltage to be output by the electrosurgical generator, in particular with regard to amplitude, frequency, curve form and/or duty cycle, wherein the curve form is preferably able to be set freely as desired. This in particular makes it possible to impress the frequency of the AC voltage generated by the inverter cells, possibly also the curve form. Advantageously, the control signal generator drives an inverter controller that is designed to drive the inverter cells such that they generate an output voltage in accordance with the reference signal. Amplitude, curve form and/or duty cycle of the generated AC voltage are furthermore in particular in accordance with the reference signal.

Provision is expediently furthermore made that the inverter cells are driven at a variable frequency. This makes it possible to react faster directly to different requirements by changing the reference signal. The frequency of the AC voltage generated by the inverter cells is thus able to be adjusted quickly, depending on the requirements of the tissue being treated by the electrosurgical instrument. It is also thus possible to change quickly and harmonically between different types of modulation.

The inverter controller is preferably designed as a high-speed controller. It is designed to generate drive signals for the inverter cells at a frequency of at least 150 MHz, preferably 200 MHz. This makes it possible to minimize distortions of the output signal. In order to be able to provide the drive signals at such a high speed, the inverter controller is preferably configured as a field-programmable gate array (FPGA).

Provision is expediently made for an output transformer on the output line, in particular in the region of the output port, of the electrosurgical generator. This serves as a galvanic isolation device in order thus to provide further certainty that the AC voltage output at the output for the connection of the electrosurgical instrument is potential-free in order to protect users and also the patient. The output transformer may in particular be configured as an output transformer unit and thus operate as a main voltage amplifier. A series capacitor is preferably additionally arranged on the output port on the secondary side of the output transformer. This acts as a DC current blocker (blocking capacitor) and thus prevents harmful DC current from flowing into the electrosurgical instrument and from there to the patient.

Advantageously, provision is made, at the end of the output line, for a low-pass filter, which is preferably configured as an at least second-order filter, in particular as an LC filter. The low-pass filter makes it possible to eliminate the high-frequency interference resulting from the high switching frequency of the inverter cells. The filter is expediently designed such that its resonance peak lies in the region between the maximum frequency for the output AC voltage and the effective switching frequency of the inverter cells. A second-order filter here makes it possible to achieve sufficient smoothing of the signal at the output of the electrosurgical generator. The low-pass filter may preferably be configured in two parts (two stages), wherein advantageously one stage is arranged upstream and one stage is arranged downstream of the output transformer. The advantages of smoothing that is close to the source are thus combined with those of smoothing that is close to the output and thus final.

In filters, there is generally a risk of the resonant frequency of the filter being excited by high-frequency components in the control signal, non-linear entities in the system or (sudden) changes in the load impedance. In order to avoid this, provision is made for a damping device that is expediently designed as an active damping device. This makes it possible to achieve sufficient damping for the low-pass filter, specifically without the undesirable power losses of the output signal that accompany passive damping measures. It is pointed out that a bandpass filter may also function in this sense as a low-pass filter provided that the upper limit frequency of the passband is high enough to eliminate the high-frequency interference.

According to one particularly advantageous embodiment, which is possibly worthy of independent protection, the active damping device comprises a feedback system that preferably has at least one current sensor on the low-pass filter. If this is an LC filter, then the current sensor is expediently arranged in series on an output port of the low-pass filter. This makes it possible to perform active damping by measuring the current actually flowing through the capacitor of the LC filter. This considerably improves impulse behavior, since the filter is able to be tuned more accurately through such active damping than in the case of conventional passive damping. It will be understood that other variables (state variables) may be added, making it possible to achieve even finer tuning of the filter. By way of example, provision may for this purpose be made for a second current sensor that is designed to determine a current at the output. Provision is preferably made for a transverse current detector for the feedback system, using which it is possible to determine (possibly parasitic) current flow in the filter and/or transformer. It is particularly expedient for a respective sensor to be arranged upstream and downstream of the transformer. This makes it possible to detect and possibly compensate for current losses caused by parasitic transverse capacitances, in particular in the transformer at the output. “Transverse” is understood here to mean a current flow or a capacitance between the two AC voltage conductors of the output line or the output of the electrosurgical generator.

The active damping device is preferably configured such that it acts on the multilevel inverter with an output signal, in particular is coupled into the driving of the inverter cells. In this case, the driving of the inverter cells is overlaid with an appropriate correction signal, thereby accordingly influencing the power output by the inverter cells. The output signal from the damping device may thus act directly on the power source in order thus to counter the occurrence of unwanted oscillations and/or unwanted impulse behavior to some extent. In technical terms, that is advantageously achieved such that the output signal from the damping device is applied to the driving of the inverter cells, and a reference signal for the inverter driving is modified, from which in turn appropriately modified drive signals for the current valves of the inverter cells are determined. The driving of the inverter cells is thus modified dynamically by the active damping device. The output voltage of the multilevel inverter is thereby controlled in a manner dependent on the output signal from the damping device.

In a further advantageous embodiment of the invention, provision is preferably made for a further output, and the multilevel inverter is furthermore designed to generate a further AC voltage that is applied to the further output. The further AC voltage expediently has a lower frequency than the high-frequency AC voltage at the output for the connection of the electrosurgical instrument. This lower frequency is preferably in the ultrasonic range. The usage spectrum of the electrosurgical generator is thus expanded considerably, since ultrasonic surgery instruments may thus also be connected and operated. This opens up the possibility for the surgeon of changing to an ultrasonic surgery instrument when needed with very little effort, without a whole other generator having to be provided and put into service for this purpose. It is also possible to use instruments that are operated simultaneously with ultrasound and high frequency.

Provision is advantageously made for at least one changeover device that is designed to selectively connect the multilevel inverter to one of the outputs. This allows the surgeon to change the output quickly and swiftly, specifically including in an intraoperative manner. It is thus possible to optimally adapt the surgical instrument quickly and easily to the patient-specific requirements depending on the conditions specifically discovered in situ.

Expediently, the inverter cells are divided in terms of circuitry on the multilevel inverter, wherein at least one portion of the inverter cells is provided for connection to the at least one further output and another portion of the inverter cells furthermore supplies the (first) output. This makes it possible to operate the further output at the same time, such that, as a result, it is possible to operate both an electrosurgical instrument at the (first) output and an ultrasonic surgical instrument at the further output. The standalone inverter cell, as it were, for the further output furthermore offers the advantage that—apart from the different frequency of the output AC voltage—it makes it possible to adapt the further AC voltage output at the further output to other voltage or current requirements of the ultrasonic surgical instrument. It is thus also possible to safely and reliably drive ultrasonic surgical instruments having different characteristics, for example having a greatly different internal resistance.

The invention is explained in more detail below by way of example with reference to advantageous embodiments. In the figures:

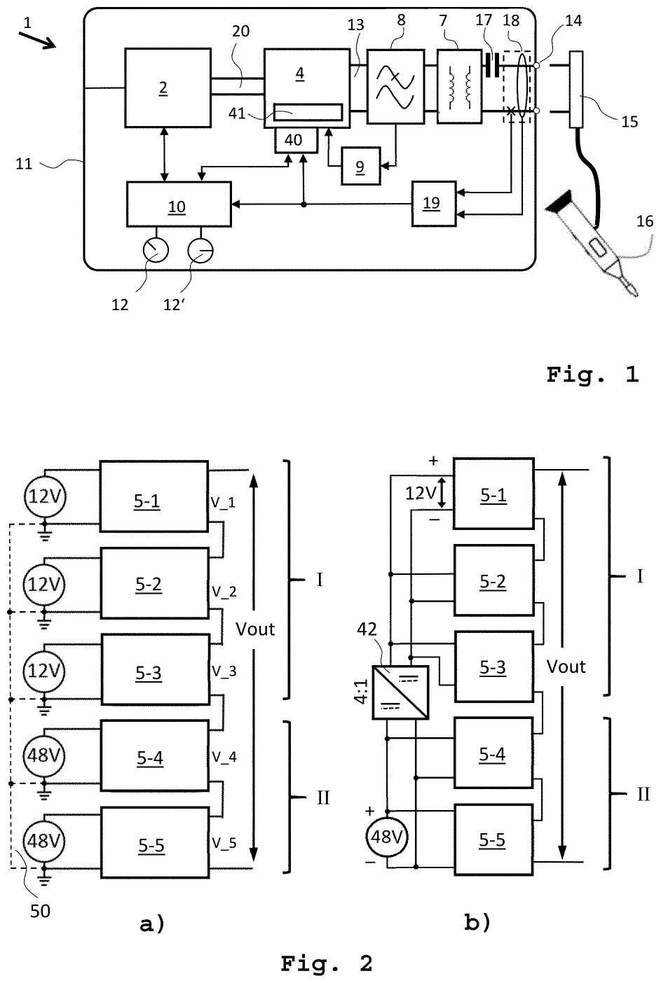

FIG. 1 shows a schematic illustration of an electrosurgical generator according to one exemplary embodiment with a connected electrosurgical instrument;

FIG. 2a, b show block diagrams of exemplary embodiments for a multilevel inverter of the electrosurgical generator according to FIG. 1 with cascaded inverter cells;

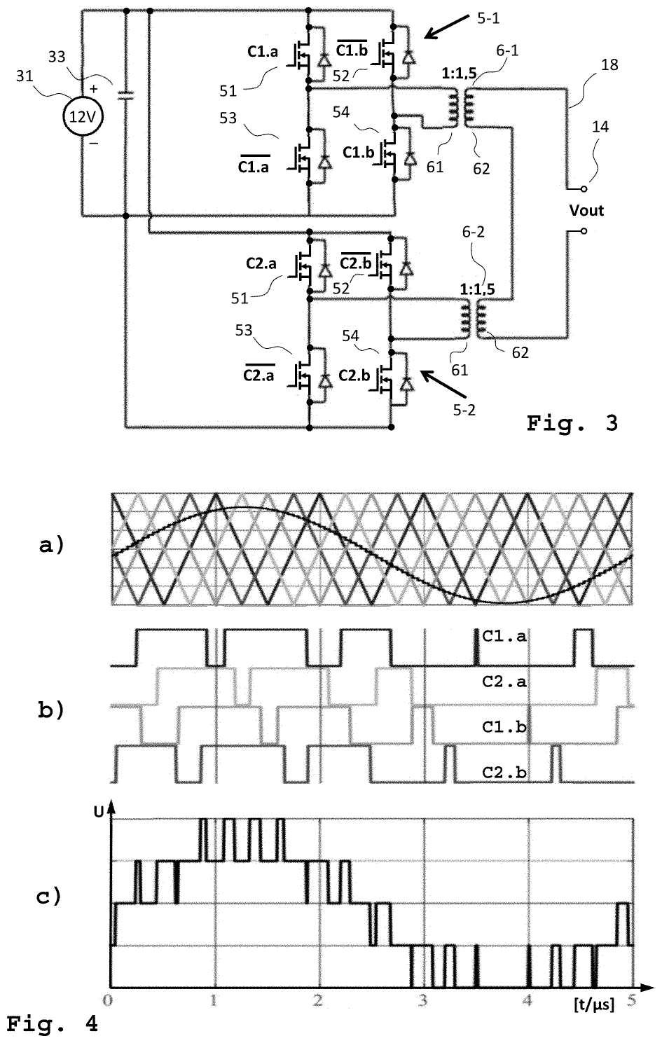

FIG. 3 shows a schematic circuit diagram of two of the inverter cells;

FIG. 4a-c show diagrams of voltage and signal profiles for switching elements of the two inverter cells according to FIG. 3;

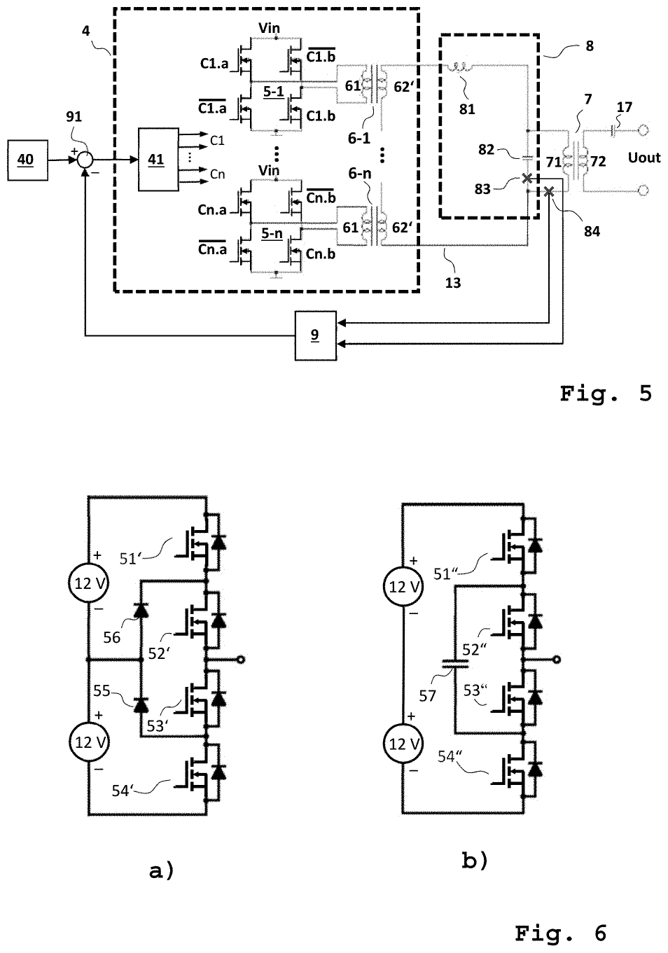

FIG. 5 shows an exemplary circuit diagram of the multilevel inverter having a plurality of cascaded inverter cells;

FIG. 6a, b show schematic circuit diagrams of alternative embodiments of the inverter cell;

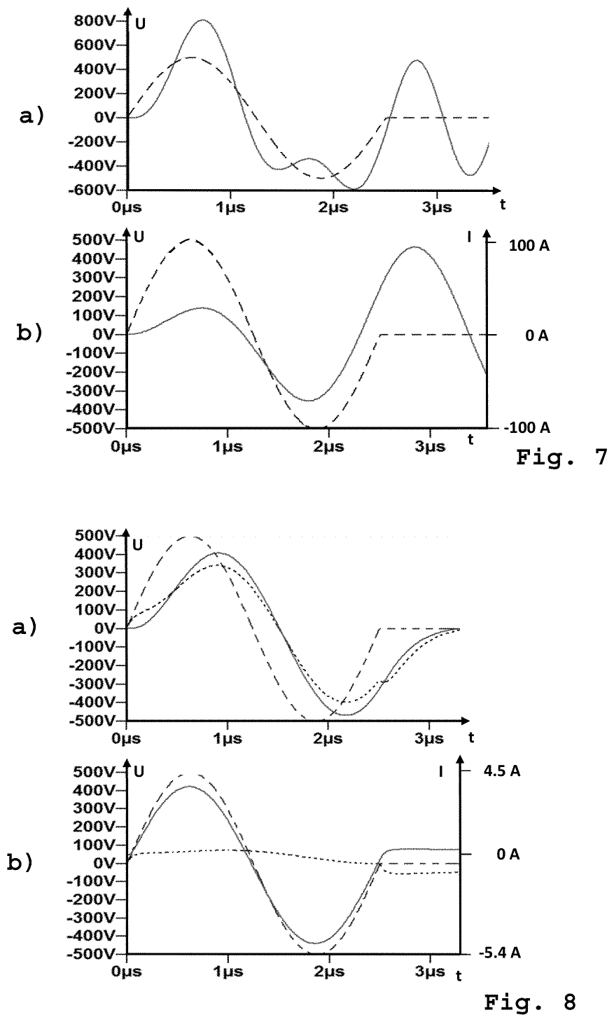

FIG. 7a, b show voltage profiles at the output without feedback in the case of a high-resistance load or short circuit;

FIG. 8a, b show voltage profiles at the output with feedback in the case of a high-resistance load or short circuit;

FIG. 9a-e show an illustration of various voltage/time profiles in high-frequency surgery;

FIG. 10 shows a schematic illustration of an electrosurgical generator according to another exemplary embodiment;

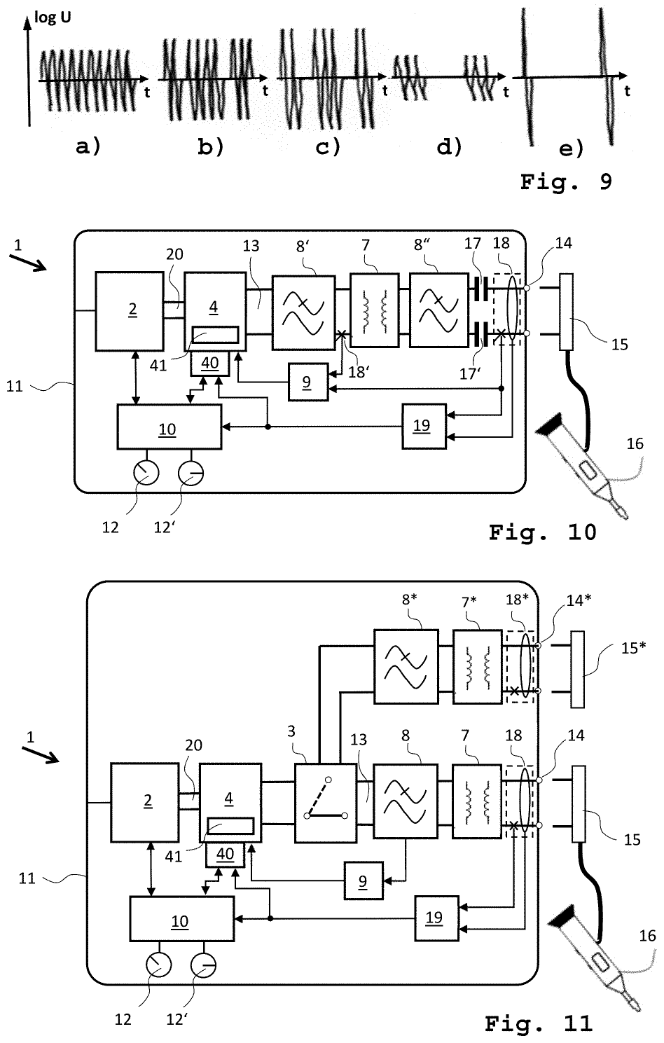

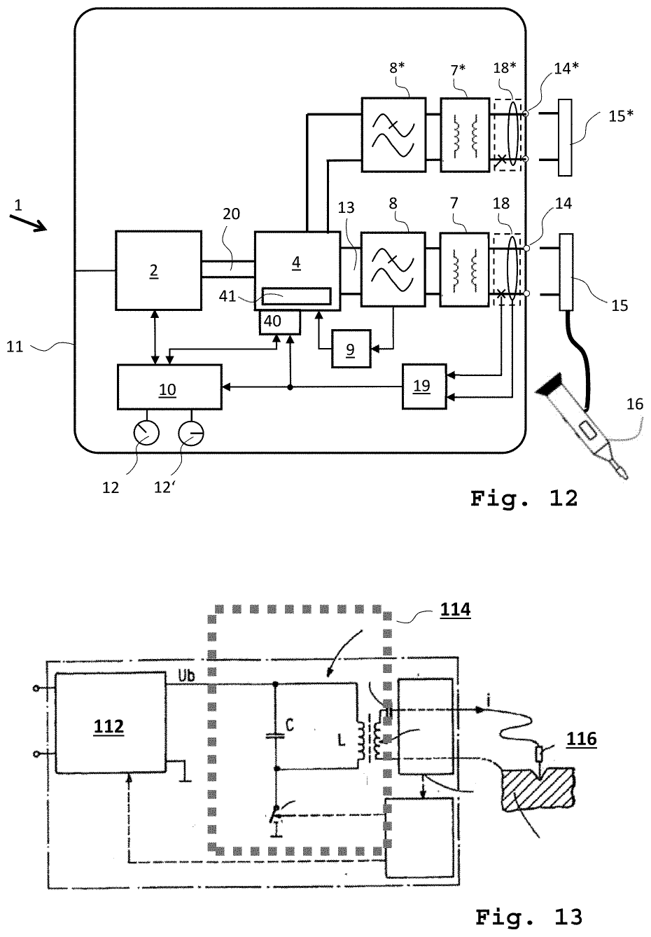

FIG. 11 shows a schematic illustration of an electrosurgical generator according to a further exemplary embodiment;

FIG. 12 shows a schematic illustration of a variant of the further exemplary embodiment according to FIG. 11; and

FIG. 13 shows a circuit diagram of an inverter according to the prior art.

An electrosurgical generator according to one exemplary embodiment of the invention is illustrated in FIG. 1. The electrosurgical generator, referenced in its entirety by the reference numeral 1, comprises a housing 11 that is provided with a port 14 for an electrosurgical instrument 16; in the illustrated exemplary embodiment, this is an electrical scalpel. It is connected to the port 14 of the electrosurgical generator 1 via a connection plug 15 of a high-voltage connection cable. The power output to the electrosurgical instrument 16 may be changed via a power controller 12.

In order to supply power to the electrosurgical generator 1, provision is made for a DC voltage supply 2, which is able to be connected, via a mains connection cable (not illustrated), to the public grid and is fed therefrom. The DC voltage supply 2 in the illustrated exemplary embodiment is a high-voltage power supply unit (High Voltage Power Supply—HVPS). It comprises a rectifier and feeds a DC link circuit 20 with DC voltage, the value of which is preferably fixed and is for example 48 volts. However, it should not be ruled out that the DC voltage value is variable between 0 and around 400 volts, wherein the absolute value of the DC voltage may in particular depend on the set power, the type of electrosurgical instrument 16 and/or its load impedance, which in turn depends on the type of tissue being treated. However, an internal power supply unit is not necessary, meaning that the DC voltage supply may also be implemented by an external power supply unit, or provision is made for a direct DC feed, for example 24 volts in vehicles or 48 volts in stationary applications.

An inverter is fed by the DC link circuit 20 and generates, from supplied DC voltage, high-frequency AC voltage in the high-voltage range of a few kilovolts, at predefinable frequencies in the range between 200 kHz and 4 MHz. The inverter is designed in the structural form of a multilevel inverter 4, as will be explained in even more detail below. The frequency and curve form of the high-frequency AC voltage to be generated by the multilevel inverter 4 are in this case predefined by an inverter controller 41 on the basis of a reference signal generated by a control signal generator 40. The high-frequency high voltage generated by the multilevel inverter 4 is routed via a low-pass filter 8 and an output transformer 7, operating as an output transformer unit for stepping up the voltage, secured against undesirable DC current components by a blocking capacitor 17 arranged in series, and output at the port 14 in the form of Uout for connection to the electrosurgical instrument 16. The voltage and current of the high voltage generated and output by the multilevel inverter 4 are furthermore measured by way of a combined voltage and current sensor 18, and the measured signals are supplied to a processing unit 19, which applies the corresponding data about the output voltage, current and power as feedback to the control signal generator 40 and to an operating controller 10 of the electrosurgical generator 1. The power controller 12 is also connected to the operating controller 10. The operating controller 10 is furthermore designed to set various what are known as modes, which are typically stored voltage/time profiles. Provision is made for a selection switch 12′ for the user to select the mode. The operating controller 10 furthermore interacts with the control signal generator 40, which is designed to generate the reference signal for the AC voltage to be output, in particular with regard to amplitude, frequency, curve form and duty cycle.

The multilevel inverter 4 comprises a plurality of series-connected inverter cells 5 that are driven by an inverter controller 41. Reference is now made to FIG. 2a. In the exemplary embodiment illustrated there, a DC voltage source having a defined DC voltage is connected to the input (illustrated on the left in the drawing) of each of the inverter cells 5. The respective inverter cell 5 generates therefrom an AC voltage that is output at the output (illustrated on the right in the drawing) of the respective inverter cell 5 in the form of AC voltage. The number of inverter cells is not limited and is as desired per se. The inverter cells 5 are numbered consecutively in FIG. 2a with the designation “5-1”, “5-2” to “5-5”, wherein the number 5 is an example and any number of at least two inverter cells may be provided. The DC voltages applied at the input of the respective inverter cell 5 are optionally coupled in terms of potential via a busbar 50. The AC voltage output at the output of the respective inverter cell 5 is accordingly denoted “V_1”, “V_2” up to “V_5”. A series connection of the inverter cells 5 results in their output voltages being added, ultimately giving, as overall output voltage:

$${V}_{o u t} = {\sum }_{i = 1}^{N} V\_i$$

The number of voltage levels able to be achieved with the “N” inverter cells 5 is in this case at least

2N+1

assuming that the DC voltages “Vin_1”, “Vin_2” to “Vin_N” applied at the input of the inverter cells 5 are all of the same value. This thus results, for example in the case of a number of five inverter cells 5, in a total of eleven possible voltage levels for the overall output voltage Vout.

The number of voltage levels may be increased considerably for an identical number of inverter cells 5 when they are fed at least in groups with DC voltage of different values. Such a configuration is shown in FIG. 2a, b. Two groups of inverter cells are formed there: a first group I containing three inverter cells 5-1, 5-2 to 5-3, which are fed from a DC voltage source with a low DC voltage, in the example 12 V; and a second group II containing two inverter cells 5-4, 5-5, which are fed from another DC voltage source with a higher DC voltage, in the example 48 V. The first group I contains low-voltage inverter cells, and the second group II contains high-voltage inverter cells. The outlay in terms of DC voltage sources is increased here, because two are now required instead of one. However, to make up for this, the number of voltage levels is increased considerably, specifically starting from eleven to more than twice that with 23 voltage levels. The number of voltage levels thus able to be achieved follows the formula

2*(mHVc*r+nLVc)+1,

wherein mHVc represents the number of higher-DC-voltage inverter cells (in the above example m=2), nLVc represents the number of low-DC-voltage inverter cells (in the above example n=3) and r represents the ratio of higher to low DC voltage (in the above example r=4).

The two voltage sources do not need to be isolated from one another in terms of potential here, but rather they may share a common reference potential, as implemented in FIG. 2a by way of the busbar 50. This also makes it possible to generate the lower DC voltage from the higher DC voltage, which may be for example the DC voltage in the link circuit 20, by way of a DC-to-DC converter 42, in particular a DC-to-DC buck converter. In the present example, as illustrated in FIG. 2b, it would be designed for a ratiometric supply with a transmission ratio of 4:1. One advantage of this configuration by way of a ratiometric supply is that changes or fluctuations in the higher DC voltage are then reflected proportionally in the lower DC voltage, meaning that the relative gradation is maintained. This makes it possible for example to increase the output voltage by 12 V in two different ways: one conventionally by activating a further 12 V inverter cell, or by activating a 48 V inverter cell in combination with deactivating three 12 V inverter cells.

The structure of the individual inverter cells 5 and their interaction are illustrated by way of example in the schematic circuit diagram according to FIG. 3. A total of two inverter cells 5-1 and 5-2 are shown there in a cascaded arrangement, in order thus also to illustrate their mutual interconnection. The common DC voltage source 31, having a supply voltage Vin of 12 volts, is illustrated on the left-hand edge of the image. It is assigned a stabilization capacitor 33. These supply the two inverter cells 5-1 and 5-2. Reference is first of all made below to the switching of the inverter cell 5-1.

Provision is made for four power switches that operate as current valves and are arranged in an H-bridge configuration. The power switches are power semiconductor switches, for example configured as IGBTs, MOSFETs or GaNFETs. The power switches 51, 53 are connected in series and form a first branch, and the power semiconductors 52, 54 are likewise connected in series and form a second branch. The center taps of the two branches are guided out and connected to both ends of a primary winding 61 of a first transformer 6-1. The transformer 6-1 furthermore has a secondary winding 62 and is used for potential isolation, wherein it may optionally furthermore have a transmission ratio for pre-amplifying the voltage; this is 1:1.5 in the illustrated example (it is pointed out that a different transmission ratio may be provided, for example with a transmission ratio of 1:1, in particular when no pre-amplification is intended to be achieved. An output line 13 is connected to the secondary winding 62 and leads to the output 14 of the electrosurgical generator 1 (possibly via a low-pass filter, not illustrated in FIG. 3).

The two power switches 51, 53 of the first branch are driven by a common signal C1.a, wherein this signal is supplied to the power switch 53 in inverted form. The two power switches 52, 54 of the second branch are accordingly likewise driven by a common signal C1.b, wherein this signal is supplied to the power switch 52 in inverted form. This means that, in the event of a HIGH signal of C1.a, the power switch 51 is put into the on state and the power switch 53 is put into the off state, that is to say the first power branch applies a positive potential to the upper connection of the primary winding 61 of the transformer 6-1. Accordingly, in the event of a HIGH signal of C2.b, the power switch 54 is put into the on state, while the power switch 52 is put into the off state in the second power branch. The second power branch thus applies a negative potential to the lower connection of the primary winding 61. In the event of a LOW signal of C1.a or C1.b, this accordingly applies vice versa, that is to say the polarity at the primary winding 61 is reversed. An AC voltage is thus generated by the inverter cell 5-1 and applied to the primary winding 61 of the transformer 6-1.

The second inverter cell 5-2 has an identical structure, and is supplied from the DC voltage source 31 in the same way as the first inverter cell 5-1. The same reference numerals are therefore used for identical elements in the figure. It is driven by the control signals C2.a and C 2.b in a manner corresponding to that described above. It thus likewise outputs, at its output, an AC voltage that is applied to a primary winding 61 of a second transformer 6-2. Since the two inverter cells 5-1 and 5-2 are fed from the same DC voltage source 31, they are connected in terms of potential. This means that the AC voltages output directly by the inverter cells 5-1 and 5-2 are not readily able to be added, since they are linked to one another in terms of their potential. However, since this output AC voltage is supplied to each of the transformers 6-1 and 6-2, the AC voltages output by the transformers 6-1 and 6-2 are each potential-free and are readily able to be added to one another to give a common output voltage that is applied to the output line 13.

The switching behavior of the power switches 51 to 54 under the effect of the control signals C1.a, C1.b, C2.a and C2.b, as are generated by the inverter controller 41 for example by way of PWM control, which is known per se, is illustrated in FIG. 4. FIG. 4a shows the obtaining of the control signals C1.a, C1.b, C2.a and C2.b. The inverter controller 41 provides a sawtooth-shaped carrier signal having a frequency of 1 MHz for each of the control signals, which are phase-offset equally from one another by 90°. These four carrier signals are illustrated by four offset sawtooth profiles in FIG. 4a. Also illustrated is the modulation signal required for the PWM modulation, in this case formed by the reference signal in the form of a sinusoidal oscillation having a frequency of 200 kHz. The signal sequences resulting from the modulation for the four control signals C1.a, C1.b, C2.a and C2.b, as are output by the inverter controller 41 for the inverter cells 5-1 and 5-2, are illustrated in FIG. 4b. These are pure rectangular-wave signal sequences that each know only a 1-bit switching state. If the power switches 51 to 54 of the two inverter cells 5-1 and 5-2 are driven with these signal sequences for the control signals in the manner described above, and the voltages respectively output by the two inverter cells 5-1 and 5-2 are added by the transformers 6-1 and 6-2, then this results in the voltage profile ultimately illustrated in FIG. 4c at the output 14. An approximately sinusoidal output voltage having five voltage levels is thus generated from the four 1-bit control signals.

An exemplary circuit diagram of the multilevel inverter 4 and its connection to adjacent components is illustrated in FIG. 5. It is possible to see the multilevel inverter 4 with its multiplicity of inverter cells, illustrated by the inverter cell 5-1 up to the inverter cell 5-n. They apply the AC voltage that they generate in each case to primary windings 61 of the transformers 6-1 to 6-n assigned thereto. In this exemplary embodiment, the transformers are configured such that their secondary windings 62′ have a higher number of turns than the primary winding 61. They are therefore designed as combined transformers and transformer units and thus ensure not only potential decoupling but also additional voltage amplification. The secondary windings 62′ are connected in series, such that their amplified voltages sum to give an increased overall voltage.

The overall voltage is output to the output line 13, at the end of which the low-pass filter 8 is arranged. This is configured as a second-order filter and comprises an inductor 81 and a capacitor 82 connected in series therewith. It is pointed out that stray inductances of the transformers 6-1 to 6-n also contribute to the inductance of the inductors 81 of the low-pass filter, and may possibly at least partially replace them. The low-pass filter 8 is tuned such that interference in the generated AC voltage due to the switching frequency of the power switches in the inverter cells of the multilevel inverter 4 is filtered out. The output of the low-pass filter 8 is applied to a primary winding 71 of an output transformer 7, which brings about galvanic isolation of the port 14 connected to the secondary winding 72. Provision is furthermore made for a blocking capacitor 17. This serves for preventing the output of DC current components to the surgical instrument 16.

The low-pass filter 8 is provided with active damping. This comprises a feedback system 9 to which the current sensor 83 is connected at input. The current sensor 83 is arranged in the same branch as the capacitor 82 of the low-pass filter 8 and thus defines the current flow through the capacitor 82. By defining the current, an appropriate signal proportional to the measured current is able to be fed back through the feedback system 9. This implements a transfer function that is selected depending on the desired behavior of the low-pass filter 8, which is now actively damped. In the simplest case, the transfer function may be configured as a proportional member. The output signal from the feedback system 9 is switched onto a negative input of a differential member 91 in order to modify the reference signal that is generated by the control signal generator 40 and connected to the positive input of the differential member 91. The reference signal modified in this way is output at the output of the differential member 91 and is applied to an input of the inverter controller 41 as drive signal for the multilevel inverter 4. The output voltage of the multilevel inverter 4 is thereby able to be controlled in a manner dependent on the feedback system 9. Undesirable resonances are thus already able to be prevented to some extent. Provision may furthermore alternatively or additionally be made for a current sensor 84 that is arranged on the primary-side port of the output transformer 7 or in series with the blocking capacitor 17 and thus defines the current flow through the output transformer 7. By defining the current, an appropriate signal proportional to the measured current is likewise able to be fed back through the feedback system 9. The feedback system implements an (appropriately expanded) transfer function that is selected according to the desired behavior, which is now actively damped, of the LC filter formed by the inductor 81 and the blocking capacitor 17.

The effect of the feedback system 9 on the voltage and current profiles at the output 14 is illustrated in FIGS. 7a, b and FIG. 8a, b. In both cases, the multilevel inverter 4 generates a pulsed AC voltage signal consisting of an individual sinusoidal oscillation (as illustrated in FIG. 9e). In the case illustrated in FIG. 7a, the load at the output is assumed to be high-resistance (in the region of 100 kOhm). The output voltage (dashed line) of the sinusoidal oscillation generated by the inverter cells 5 of the multilevel inverter 4 is thereby additionally overlaid with a resonant oscillation. This results from the resonant frequency of the LC filter formed by the inductors 81 and the capacitor 82 in accordance with the known formula

$$f = \frac{1}{2 \cdot π \sqrt{L \cdot C}} .$$

The resultant overlaid output signal is illustrated by the solid line. It is possible to see a considerable deformation of the curve and pronounced reverberation. The complementary case of a short circuit is illustrated in FIG. 7b. It is again possible to see the (identical) sinusoidal oscillation generated by the inverter cells 5 of the multilevel inverter 4 (dashed line). In addition to this, there is an overlap from the resonant oscillation, which results from the filter inductor 81, which resonates with the blocking capacitor 17 at the output 14. The current profile that results in this case is illustrated with the solid line at the output 14. It may be seen that a considerable interfering oscillation builds up.

The same cases are illustrated in FIG. 8a, b, wherein the filter 8 is damped by way of the feedback system 9. FIG. 8a again shows the case with a high-resistance load. The original output signal from the inverter cells 5 of the multilevel inverter 4 is also illustrated with a dashed line as reference. The actual output signal overlaid with interference is fed back via the feedback system 9 using measured signals from the current sensor 83 and changes the signal supplied to the multilevel inverter 4 by acting on the differential member 91. This reference signal is bent in a targeted manner, as it were, giving rise to a modified reference signal, which is actually applied to the inverter controller 41 as control signal in order to drive the inverter cells 5. The resulting output signal (see dashed line, which illustrates a smoothed profile) is “bent” in a targeted manner such that the overlaid oscillation at the output is counteracted in a targeted manner. The actual output signal ultimately resulting from the generated voltage, which is “bent” in a targeted manner, of the multilevel inverter 4 and from the resonant oscillation of the filter 8 is illustrated by the solid line. It is readily able to be seen through comparison with FIG. 7a that the actual output signal is a substantially more harmonic sinusoidal oscillation.

The same applies to the short-circuit case using the feedback system 9. This case is illustrated in FIG. 8b. The original drive signal, as generated as reference signal by the control signal generator 40, is again illustrated with a dashed line. The modified reference signal ultimately generated under the effect of the feedback system 9 using measured signals from the current sensor 84 is used to drive the inverter cells 5.

The resulting output signal is illustrated (following smoothing) by the dashed line. It is surprisingly small in relation to the voltage amplitude, the reason for which is that the undesirable resonant frequency lies very close to the frequency of the AC voltage generated by the multilevel inverter 4. Only a very small actual drive signal for the inverter controller 41 is thus required. The actual current profile that then results at the output 14 is again illustrated with the solid line. It is readily able to be seen through comparison with FIG. 7a that the actual output signal is a substantially more harmonic sinusoidal oscillation. It may clearly be seen, through comparison with FIG. 7b, that the actual sinusoidal oscillation is reproduced significantly more accurately (interval up to 2 μs) and parasitic reverberation is then effectively suppressed (no “ringing” effect). The feedback using the measured signals from the current sensor 84 thus ensures a considerably better and lower-harmonic sinusoidal output signal in spite of the critical LC filter 8 with the blocking capacitor 17 at the output 14.

As a result, the multilevel inverter 4 according to the invention may be used to finely and precisely predefine the AC voltage profiles to be output. The multilevel inverter 4 driven by the reference signal in particular gives full control of the curve form, specifically in particular including in the case of modulated output signals. Modulated output signals are thus able to be generated accurately and in a reproducible manner, as illustrated in FIGS. 9a to 9e. In order to ensure a constant energy output, the multilevel inverter 4 according to the invention furthermore makes it possible, in highly modulated modes with a shorter duty cycle, to increase the value of the output voltage to the extent that, in spite of the short switch-on time, the same energy is output to the electrosurgical instrument 16 as in the modes with a longer switch-on time or in the continuous mode.

The invention thus allows more dynamic and more accurate control of the output high-frequency AC voltage, specifically including and specifically in pulsed modes. The modes are again able to be kept considerably more precise thanks to the optional feedback.

It is furthermore pointed out that the invention is not restricted to inverter cells 5 with an H-bridge configuration. Provision may also be made for other topologies for the inverter cells 5. FIGS. 6a and 6b show examples of these and illustrate alternative topologies, specifically likewise each having four switching elements 51′ to 54′ and 51″ to 54″. FIG. 6a thus shows a configuration of the inverter cell with a type of structure with neutral point clamping by way of diodes 55, 56, and FIG. 6b with a type of structure with a floating capacitor 57. Similarly to the inverter cells in an H-bridge configuration, these may likewise be cascaded in order to achieve a higher number of voltage levels.

FIG. 10 illustrates an alternative exemplary embodiment to the exemplary embodiment according to FIG. 1. Elements that are identical or of the same type are denoted using the same reference numerals. It differs essentially in that the low-pass filter 8 has a two-stage configuration in the alternative exemplary embodiment. A first stage 8′ of the low-pass filter is furthermore arranged directly at the output of the multilevel inverter 4 in order to smooth the generated AC voltage. A second stage 8″ of the low-pass filter is arranged on the output side of the output transformer 7. Further smoothing thus takes place just before the output, in order in particular also to detect interference caused by the output transformer 7. It is pointed out that the stray inductance of the output transformer 7 may also contribute to the inductance of the inductors 81 of the second stage 8″ of the low-pass filter, and may possibly at least partially replace them.

In the embodiment according to FIG. 10, provision is made for dual blocking capacitors 17, 17′ for increasing safety. It will be understood that such a dual arrangement may also be provided in the other exemplary embodiments.

An expedient alternative arrangement of the current sensors for the feedback system is also illustrated using the example of this exemplary embodiment according to FIG. 10; this may also be provided in the other exemplary embodiments. Provision is made in this case for a current sensor 18′ in series on the low-pass filter 8, more precisely on the output of the first stage 8′. The combined current and voltage sensor 18 functions as second current sensor. Based on these signals, it is possible to measure the actual output current (which is transmitted to the operating controller 10 via the processing unit 19) along with the current flow on the input side of the output transformer 7. A transverse current detector is also formed. This is designed to determine, from a current difference that results here, the magnitude of a current through a capacitor 82 of the low-pass filter (here the second stage 8″ of the low-pass filter). This may be acquired by the feedback system 9 and compensated for by changing the driving of the multilevel inverter 4. It is thereby also possible to detect and compensate for current losses caused by parasitic transverse capacitance that is not otherwise able to be measured directly, in particular of the output transformer 7 or of the low-pass filter 8 with its stages 8′, 8″.

A further exemplary embodiment of an electrosurgical generator according to the present invention is illustrated in FIG. 11. This is based on the exemplary embodiment illustrated in FIG. 1, but differs therefrom in that provision is made for a second output 14* and a changeover device 3. The multilevel inverter 4 is connected to the input of said changeover device and the output line 13 is connected to one of its outputs and leads, via the low-pass filter 8 and the output transformer 7, to the (first) output 14 for the electrosurgical instrument 16. A second output 14* is connected to the other output of the changeover device 3 via a second output line 13*, a second low-pass filter 8* and a second output transformer 7′. A connection plug 15* for a second instrument (not illustrated) may be connected to said second output, wherein the second instrument may be in particular an ultrasonic surgical instrument, such as for example an ultrasonic scalpel. Provision is made for another at least one blocking capacitor 17 (not illustrated) at each of the outputs 14, 14*, as in the exemplary embodiment shown in FIG. 1.

The changeover device 3 is designed to output the AC voltage generated by the multilevel inverter 4 selectively at the output 14 to the instrument 16 connected there, in particular the electrosurgical instrument 16, or at the output 14* to the instrument connected there, in particular the ultrasonic surgical instrument. Using the same electrosurgical generator 1, it is thus possible, as the surgeon wishes, to use an electrosurgical instrument, such as for example an electrocauter, or an ultrasonic surgical instrument, such as for example ultrasonic dissecting scissors. The change between the instruments is made considerably easier and may even take place in an intraoperative manner. The field of application for the electrosurgical generator is thus broadened considerably. As an alternative or in addition, in one variant as illustrated in FIG. 12, provision may also be made for the inverter cells 5 to be divided in terms of circuitry. In this case, at least one (but not all) of the inverter cells 5 is connected to the second output 14* and is able to supply same for example with an AC voltage in the ultrasonic frequency range, while the rest of the inverter cells 5-1 to 5-4 continue to supply the output 14 with high-frequency AC voltage. It is thereby also possible to operate two electrosurgical instruments in parallel (including in different modes), or it is also readily possible to operate an instrument that uses both ultrasound and high-frequency energy.

1. An electrosurgical generator that is designed to output a high-frequency AC voltage to an electrosurgical instrument, comprising a DC voltage supply and a high-voltage inverter that is fed from the DC voltage supply and generates a high-frequency AC voltage having a variable voltage and frequency that is applied to an output for the connection of the electrosurgical instrument, wherein
the inverter is configured as a multilevel inverter and comprises a plurality of inverter cells connected in a cascaded manner that are driven by a control device.

2. The electrosurgical generator as claimed in claim 1, wherein the inverter cells have potential decoupling at output.

3. The electrosurgical generator as claimed in claim 2, wherein a respective transformer is connected at the output of the respective inverter cell with its primary side.

4. The electrosurgical generator as claimed in claim 3, wherein the transformers are each provided with a transformer unit as preamplifier for stepping up the voltage.

5. The electrosurgical generator as claimed in claim 1, wherein the inverter cells are fed from in each case one voltage source.

6. The electrosurgical generator as claimed in claim 1, wherein a plurality of, at least two groups of inverter cells are provided, wherein the inverters of the respective group are supplied jointly by one DC voltage source.

7. The electrosurgical generator as claimed in claim 1, wherein a plurality of, at least two groups of inverter cells are provided, wherein the groups are supplied with DC voltage of different values.

8. The electrosurgical generator as claimed in claim 7, wherein provision is made for in each case at least one DC-to-DC converter for supplying at least one of the groups with a different voltage.

9. The electrosurgical generator as claimed in claim 1, wherein DC voltage sources for supplying the inverter cells are galvanically coupled.

10. The electrosurgical generator as claimed in claim 1, wherein the DC voltage supply is designed as a fixed voltage supply.

11. The electrosurgical generator as claimed in claim 1, wherein the inverter cells are each configured with a type of structure with neutral point clamping at their DC voltage supply or with a floating capacitor.

12. The electrosurgical generator as claimed in claim 1, wherein the inverter cells are connected in series.

13. The electrosurgical generator as claimed in claim 1, wherein
provision is made for a control signal generator for the multilevel inverter that is designed to generate a reference signal for driving the multilevel inverter.

14. The electrosurgical generator as claimed in claim 13, wherein the reference signal is a pattern for AC voltage to be output by the electrosurgical generator.

15. The electrosurgical generator as claimed in claim 13, wherein the control signal generator drives an inverter controller that is designed to drive the inverter cells such that they generate an output voltage in accordance with the reference signal.

16. The electrosurgical generator as claimed in claim 1, wherein the inverter cells are driven with a variable-frequency reference signal.

17. The electrosurgical generator as claimed in claim 1, wherein provision is made for an output transformer on the output line as a further galvanic isolation device.

18. The electrosurgical generator as claimed in claim 16, wherein provision is made, in the output line, for a low-pass filter.

19. The electrosurgical generator as claimed in claim 18, wherein provision is made for an active damping device for the low-pass filter.

20. The electrosurgical generator as claimed in claim 19, wherein the active damping device comprises a feedback system, wherein the feedback system has at least one current sensor on the low-pass filter.

21. The electrosurgical generator as claimed in claim 19, wherein an output signal from the active damping device acts on the multilevel inverter.

22. The electrosurgical generator as claimed in claim 1, wherein
provision is made for at least one further output to which a further AC voltage generated by the multilevel inverter is applied.

23. The electrosurgical generator as claimed in claim 22, wherein the at least one further AC voltage has a lower frequency than the high-frequency AC voltage at the output for the connection of the electrosurgical instrument.

24. The electrosurgical generator as claimed in claim 22, wherein provision is made for at least one changeover device that is designed to selectively connect the multilevel inverter to one of the outputs.

25. The electrosurgical generator as claimed in claim 23, wherein the inverter cells are divided in terms of circuitry such that at least one portion of the inverter cells is provided for connection to the at least one further output and another portion of the inverter cells furthermore supplies the output.
